(19) United States
(12) Patent Application Publication
(10) Pub. No.: US 2014/0303718 A1
(43) Pub. Date: Oct. 9, 2014

(54) Retrieval and repositioning system for prosthetic heart valve

(71) Applicant: Tendyne Holdings, Inc., Roseville, MN (US)

(72) Inventors: Zachary J. TEGELS, Minneapolis, MN (US); Robert M. VIDLUND, Forest Lake, MN (US)

(73) Assignee: Tendyne Holdings, Inc., Roseville, MN (US)

(21) Appl. No.: 14/154,816

(22) Filed: Jan. 14, 2014

Related U.S. Application Data
(60) Provisional application No. 61/808,458, filed on Apr. 4, 2013.

(51) Int. Cl. A61F 2/24

(52) U.S. Cl. CPC A61F 2/2427
USPC 623/2.11

(57) ABSTRACT

This invention relates to the design and function of a retrieval device for a prosthetic heart valve for re-positioning or removal of a previously implanted valve prosthesis from a beating heart without extracorporeal circulation using a transcatheter retrieval system.

### CROSS REFERENCE TO RELATED APPLICATIONS

Not applicable.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

SEQUENCE LISTING INCLUDED AND INCORPORATED BY REFERENCE HEREIN

Not applicable.

### BACKGROUND

1. Field of the Invention

This invention relates to a novel device and method for retrieval of a transcatheter heart valve replacement or for capture and repositioning of a deployed transcatheter heart valve replacement.

2. Background of the Invention

Valvular heart disease and specifically aortic and mitral valve disease is a significant health issue in the US. Annually approximately 90,000 valve replacements are conducted in the US. Traditional valve replacement surgery, the orthotopic replacement of a heart valve, is an “open heart” surgical procedure. Briefly, the procedure necessitates a surgical opening of the thorax, initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients.

Thus if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated to the native mitral valve apparatus and thus a greater level of difficulty with regards to inserting and anchoring the replacement prosthesis.

Several designs for catheter-deployed (transcatheter) aortic valve replacement are under various stages of development. The Edwards SAPIEN® transcatheter heart valve is currently undergoing clinical trial in patients with calcific aortic valve disease who are considered high-risk for conventional open-heart valve surgery. This valve is deployable via a retrograde transarterial (transfemoral) approach or an antegrade transapical (transventricular) approach. A key aspect of the Edwards SAPIEN® and other transcatheter aortic valve replacement designs is their dependence on lateral fixation (e.g. tines) that engages the valve tissues as the primary anchoring mechanism. Such a design basically relies on circumferential friction around the valve housing or stent to prevent dislodgement during the cardiac cycle. This anchoring mechanism is facilitated by, and may somewhat depend on, a calcified aortic valve annulus. This design also requires that the valve housing or stent have a certain degree of rigidity.

At least one transcatheter mitral valve design is currently in development. The Endovalve uses a folding tripod-like design that delivers a tri-leaflet bioprosthetic valve. It is designed to be deployed from a minimally invasive transatrial approach, and could eventually be adapted to a transvenous atrial septotomy delivery. This design uses “proprietary gripping features” designed to engage the valve annulus and leaflets tissues. Thus the anchoring mechanism of this device is essentially equivalent to that used by transcatheter aortic valve replacement designs.

Various problems continue to exist in this field, including problems with how to retrieve a collapsible heart valve prosthetic from the native valve once the prosthetic has reached the end of its useful life. For example, a prosthetic heart valve may be delivered and secured percutaneously or intravenously using a catheter and endoscope or similar device, but the process of disengaging anchoring mechanisms and collapsing the prosthetic for retrieval is often more difficult to accomplish than is the delivery. Accordingly, there is a need for an improved device and method for retrieval when such valves need to be replaced.

### BRIEF SUMMARY OF THE INVENTION

In one embodiment, there is provided a prosthetic heart valve retrieval device, comprising: a dilator tip with a radio band, said dilator tip mounted at distal end of a dilator sheath, said dilator sheath having a lumen therethrough and said dilator sheath mounted on a distal side of dilator base, said dilator base having a sheath lock for operatively engaging the dilator sheath, said dilator base having a slidably removable luer-lock introducer disposed within the lumen, said dilator base having a guide rod aperture for engaging a guide rod that is connected to a guide rod handle mount that is attached on top of a handle apparatus, said dilator base having a traveller strap affixed on a proximal side and said traveller strap extending proximally to engage a tensioning unit on the handle apparatus, said handle apparatus having an actuator and a spring operatively connected to the traveller strap, wherein when the actuator is engaged the traveller strap is pulled proximally through the tensioning unit and the dilator base slides along guide rod towards the handle apparatus.

In another preferred embodiment, there is provided a device wherein the dilator tip is bullet-shaped, cone-shaped, hooded, or otherwise shaped to guide the valve tether into the lumen of the dilator sheath.

In another preferred embodiment, there is provided a method of using the retrieval device for capturing a tethered expandable prosthetic heart valve to re-position or remove said valve, comprising the steps of: (i) inserting said retrieval device, containing a tethered and expandable prosthetic heart valve, into a patient, and (ii) capturing and retracting the tether into the retrieval device.

In another preferred embodiment, there is provided wherein the method may further include the step of inserting the retrieval device by directly accessing the heart through the intercostal space, or using an apical approach to enter a heart ventricle.

In another preferred embodiment, there is provided wherein the method may further include the step of inserting the retrieval device by directly accessing the heart through a thoracotomy, sternotomy, or minimally-invasive thoracic, thorascopic, or trans-diaphragmatic approach to enter the left ventricle.

In another preferred embodiment, there is provided wherein the method may further include the step of (iii) removing the tethered expandable prosthetic heart valve from the patient by collapsing the expandable prosthetic heart valve apparatus into the dilator sheath catheter and retracting the dilator sheath.

### BRIEF DESCRIPTION OF THE DRAWINGS

The attached figures provide enabling and non-limiting example of certain features of the present invention. The figures are not intended to be limiting in any way to the description that is provided in the text.

FIG. 1 is a side view of one embodiment of the prosthetic valve retrieval system provided herein.

FIG. 2 is a side view of the dilator and tip components with handle and tapered-connector (luer+tuohy borst).

FIG. 3 is a side view of one embodiment of a dilator tip.

FIG. 4 is a side view of another embodiment of a dilator tip.

FIG. 5 is a side view of yet another embodiment of a dilator tip.

FIG. 6 is a side view of the retrieval system in operation and connected to a tether of a prosthetic mitral valve.

### DETAILED DESCRIPTION OF THE INVENTION

### Functions of the Retrievable Stented Prosthetic Mitral Valve

The present invention provides in one embodiment a retrieval system for a previously deployed prosthetic heart valve wherein a valve tether is attached to the valve or to a collapsible stent containing the valve.

The invention allows for the capture of the single retrieval tether by a catheter-based extraction device, and for the re-positioning or removing the entire deployed valve apparatus via the retrieval device.

The prosthetic heart valve contemplated for retrieval using the retrieval device comprises a self-expanding tubular stent having a cuff at one end and tether loops for attaching tether(s) at the other end, and disposed within the tubular stent is a leaflet assembly that contains the valve leaflets, the valve leaflets being formed from stabilized tissue or other suitable biological or synthetic material. In one embodiment, the leaflet assembly comprises a wire form where a formed wire structure is used in conjunction with stabilized tissue to create a leaflet support structure which can have anywhere from 1, 2, 3 or 4 leaflets, or valve cusps disposed therein. In another embodiment, the leaflet assembly is wireless and uses only the stabilized tissue and stent body to provide the leaflet support structure, without using wire, and which can also have anywhere from 1, 2, 3 or 4 leaflets, or valve cusps disposed therein.

The tether anchors the valve to an anchoring location within the ventricle. Preferably, the location is the apex of the heart and uses an epicardial attachment pad. However, other tether attachment locations may be used in the deployment of the valve and also therefore, for the retrieval.

The cuff of the valve functions to counter the forces that act to displace the prosthesis toward/into the ventricle (i.e., atrial pressure and flow-generated shear stress) during ventricular filling. Accordingly, the stent containing the valve is positioned and pulled between the ventricular tether and the atrial cuff.

### Cuff Structure

The cuff is a substantially flat plate that projects beyond the diameter of the tubular stent to form a rim or border. As used herein, the term cuff, flange, collar, bonnet, apron, or skirting are considered to be functionally equivalent. When the tubular stent is pulled through the mitral valve aperture, the mitral annulus, by the tether loops in the direction of the left ventricle, the cuff acts as a collar to stop the tubular stent from traveling any further through the mitral valve aperture. The entire prosthetic valve is held by longitudinal forces between the cuff which is seated in the left atrium and mitral annulus, and the ventricular tethers attached to the left ventricle.

The cuff is formed from a stiff, flexible shape-memory material such as the nickel-titanium alloy material Nitinol® wire that is covered by stabilized tissue or other suitable biocompatible or synthetic material. In one embodiment, the cuff wire form is constructed from independent loops of wire that create lobes or segments extending axially around the circumference of the bend or seam where the cuff transitions to the tubular stent (in an integral cuff) or where the cuff is attached to the stent (where they are separate, but joined components).

Once covered by stabilized tissue or material, the loops provide the cuff with the ability to travel up and down, to articulate, along the longitudinal axis that runs through the center of the tubular stent. In other words, the individual spindles or loops can independently move up and down, and can spring back to their original position due to the relative stiffness of the wire. The tissue or material that covers the cuff wire has a certain modulus of elasticity such that, when attached to the wire of the cuff, such tissue or material allows the wire spindles to move.

The cuff counteracts the longitudinal ventricular pressure during systole against the prosthesis in the direction of the left ventricle to keep the valve from being displaced or slipping into the ventricle. The tether(s) counteracts this force and is used to maintain the valve position and withstand the ventricular force during ventricular contraction or systole. Accordingly, the entire valve must be positioned in a proper position and cannot be radially misplaced during the deployment process. After a period of time, changes in the geometry of the heart and/or fibrous adhesion between prosthesis and surrounding cardiac tissues may assist or replace the function of the ventricular tethers in resisting longitudinal forces on the valve prosthesis during ventricular contraction, so the initial deployment must be accurate.

### Stent Structure

Preferably, superelastic metal wire, such as Nitinol® wire, is also used for the stent, for the inner wire-based leaflet assembly that is disposed within the stent, and for the cuff wire form. Such stents are available from any number of commercial manufacturers, such as Pulse Systems. Laser cut stents are preferably made from Nickel-Titanium (Nitinol®), but also without limitation made from stainless steel, cobalt chromium, titanium, and other functionally equivalent metals and alloys, or Pulse Systems braided stent that is shape-set by heat treating on a fixture or mandrel.

One key aspect of the stent design is that it be compressible and when released have the stated property that it return to its original (uncompressed) shape. This requirement limits the potential material selections to metals and plastics that have shape memory properties. With regards to metals, Nitinol® has been found to be especially useful since it can be processed to be austenitic, martensitic or super elastic. Martensitic and super elastic alloys can be processed to demonstrate the required compression features.

### Laser Cut Stent

One possible construction of the stent envisions the laser cutting of a thin, isodiametric Nitinol® tube. The laser cuts form regular cutouts in the thin Nitinol tube. Secondarily the tube is placed on a mold of the desired shape, heated to the martensitic temperature and quenched. The treatment of the stent in this manner will form a stent or stent/cuff that has shape memory properties and will readily revert to the memory shape at the calibrated temperature.

### Leaflet and Inner Wireform

The valve leaflets are held by, or within, a leaflet assembly. In one preferred embodiment of the invention, the leaflet assembly comprises a leaflet wire support structure to which the leaflets are attached and the entire leaflet assembly is housed within the stent body. In this embodiment, the assembly is constructed of wire and stabilized tissue to form a suitable platform for attaching the leaflets. In this aspect, the wire and stabilized tissue allow for the leaflet structure to be compressed when the prosthetic valve is compressed within the deployment catheter, and to spring open into the proper functional shape when the prosthetic valve is opened during deployment. In this embodiment, the leaflet assembly may optionally be attached to and housed within a separate cylindrical liner made of stabilized tissue or material, and the liner is then attached to line the interior of the stent body.

In this embodiment, the leaflet wire support structure is constructed to have a collapsible/expandable geometry. In a preferred embodiment, the structure is a single piece of wire. The wireform is, in one embodiment, constructed from a shape memory alloy such as Nitinol®. The structure may optionally be made of a plurality of wires, including between 2 to 10 wires. Further, the geometry of the wire form is without limitation, and may optionally be a series of parabolic inverted collapsible arches to mimic the saddle-like shape of the native annulus when the leaflets are attached. Alternatively, it may optionally be constructed as collapsible concentric rings, or other similar geometric forms that are able to collapse or compress, then expand back to its functional shape. In certain preferred embodiments, there may be 2, 3 or 4 arches. In another embodiment, closed circular or ellipsoid structure designs are contemplated. In another embodiment, the wire form may be an umbrella-type structure, or other similar unfold-and-lock-open designs. A further preferred embodiment utilizes super elastic Nitinol® wire approximately 0.015″ in diameter. In this embodiment, the wire is wound around a shaping fixture in such a manner that 2-3 commissural posts are formed. The fixture containing the wrapped wire is placed in a muffle furnace at a pre-determined temperature to set the shape of the wire form and to impart it's super elastic properties. Secondarily, the loose ends of the wireform are joined with a stainless steel or Nitinol tube and crimped to form a continuous shape. In another preferred embodiment, the commissural posts of the wireform are adjoined at their tips by a circular connecting ring, or halo, whose purpose is to minimize inward deflection of the post(s).

### Deployment of the Retrieval Device

The retrieval device is, in one embodiment, delivered through the apex of the left ventricle of the heart. In one aspect of the apical delivery, the retrieval device accesses the heart and pericardial space by intercostal delivery.

### Tether

The tether(s) is attached to the prosthetic heart valve and extend to one or more tissue anchor locations within the heart. In one preferred embodiment, the tether(s) extend downward through the left ventricle, exiting the left ventricle at the apex of the heart to be fastened on the epicardial surface outside of the heart. In another preferred embodiment, the tether is optionally anchored to other tissue locations depending on the particular application of the prosthetic heart valve, such as one or both papillary muscles, septum, and/or ventricular wall.

The tether is made from surgical-grade materials such as biocompatible polymer suture material. Examples of such material include 2-0 exPFTE (polytetrafluoroethylene) or 2-0 polypropylene.

### DESCRIPTION OF THE FIGURES

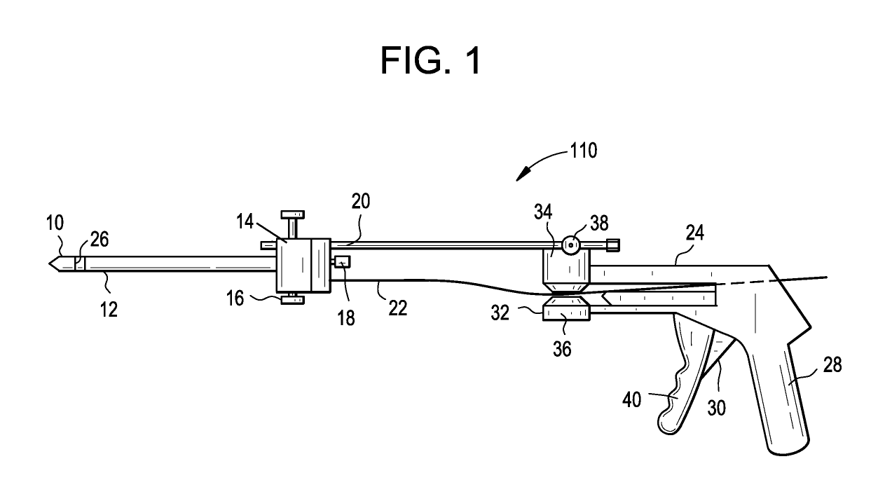

Referring now to the FIGURES, FIG. 1 is a side view of one embodiment of the prosthetic valve retrieval system provided herein. FIG. 1 shows valve retrieval system 110 having dilator tip 10 with radio band 26 mounted at the distal end of dilator sheath 12. Dilator base 14 has sheath lock 16 and luer-lock introducer 18. Guide rod 20 connects dilator base 14 to guide rod handle mount 38. Guide rod handle mount 38 sits atop tensioning unit 32 which has tensioning unit top 34 and tensioning unit bottom 36, and the tensioning unit 32 provides segmented advancement of the traveller strap 22 that is affixed to dilator base 14 and extends proximally through the tensioning unit 32 towards handle apparatus 24. Handle apparatus 24 has the tensioning unit 32 affixed at a distal end and the proximal end of handle apparatus is composed of handle 28 and actuator 40 with actuator spring 30 providing a longitudinal tensioning force on traveller strap 22. An important feature is the placement of the guide rod 20 and related assemblies on top of the handle which alleviates interference of the guide rod 20 during the retrieval process.

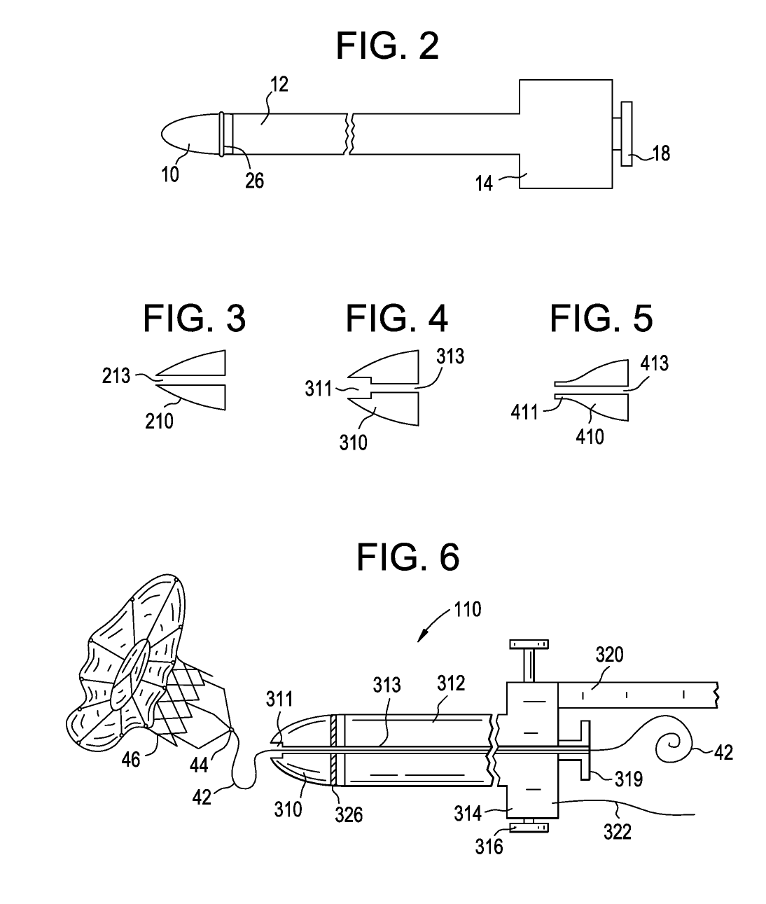

FIG. 2 is a side view of the dilator and tip components with handle and tapered-connector (luer+tuohy borst). FIG. 2 shows dilator tip 10 with radio band 26 to assist in roentgenographic imaging. Dilator sheath 12 is connected to dilator base 14 with luer-lock introducer 18 maintaining a seal to prevent intracardiac fluid/blood loss.

FIG. 3 is a side view of one embodiment of a dilator tip 210, and shows lumen 213 that is used to extend the capture wire and pull the valve tether down into the lumen 213.

FIG. 4 is a side view of another embodiment of a dilator tip 310 with tether capture lumen 313, and FIG. 4 also shows capture recess 311, which facilitates capture of a valve tether that may have a beaded or enlarged feature at the tether connection point where the tether connects to the valve.

FIG. 5 is a side view of yet another embodiment of a dilator tip 410, and shows lumen 413 that is used to extend the capture wire and pull the valve tether down into the lumen 413. FIG. 5 also shows extended tip 411 which can be used to facilitate access and/or capture/repositioning in certain circumstances.

FIG. 6 is a side view of the retrieval system 110 in operation and connected to a tether 42 of a prosthetic mitral valve 46. FIG. 5 shows dilator tip 310 with bead capture recess 311 for capturing tether connection bead 44 and captured tether 42 extending down into lumen 313 through dilator shaft 312. Radio band 326 is shown marking the dilator tip 310. Dilator shaft 312 is connected to dilator base 314 and vertically-slidable sheath lock 316 in an up, or locked position. Guide rod 320 is shown connected to dilator base 314 and traveller strap 322 is shown affixed to the dilator base. Luer-lock introducer sleeve 319 is shown with valve tether 42 exiting from lumen 313.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed is:

1. A prosthetic heart valve retrieval device, comprising: a dilator tip with a radio band, said dilator tip mounted at distal end of a dilator sheath, said dilator sheath having a lumen therethrough and said dilator sheath mounted on a distal side of dilator base, said dilator base having a sheath lock for operatively engaging the dilator sheath, said dilator base having a slidably removable luer-lock introducer disposed within the lumen, said dilator base having a guide rod aperture for engaging a guide rod that is connected to a guide rod handle mount that is attached on top of a handle apparatus, said dilator base having a traveller strap affixed on a proximal side and said traveller strap extending proximally to engage a tensioning unit on the handle apparatus, said handle apparatus having an actuator and a spring operatively connected to the traveller strap, wherein when the actuator is engaged the traveller strap is pulled proximally through the tensioning unit and the dilator base slides along guide rod towards the handle apparatus.

2. The prosthetic heart valve retrieval device of claim 1, further comprising wherein the dilator tip is bullet-shaped, cone-shaped, hooded, or otherwise shaped to guide the valve tether into the lumen of the dilator sheath.

3. A method of using the retrieval device of claim 1 for capturing a tethered expandable prosthetic heart valve to re-position or remove said valve, comprising the steps of: (i) inserting said retrieval device containing a tethered and expandable prosthetic heart valve into a patient, and (ii) capturing and retracting the tether into the retrieval device.

4. The method of claim 3, wherein the step of inserting the retrieval device by directly accessing the heart through the intercostal space, or using an apical approach to enter a heart ventricle.

5. The method of claim 3, wherein the step of inserting the retrieval device by directly accessing the heart through a thoracotomy, sternotomy, or minimally-invasive thoracic, thorascopic, or trans-diaphragmatic approach to enter the left ventricle.

6. The method of claim 3, further comprising the step of (iii) removing the tethered and expandable heart valve from the patient by collapsing the expandable prosthetic heart valve apparatus into the dilator sheath catheter and retracting the dilator sheath.
